(19) United States
(12) Patent Application Publication
(10) Pub. No.: US 2024/0180421 A1
(43) Pub. Date: Jun. 6, 2024

(54) METHOD FOR DETERMINING AN EYE POSITION

(71) Applicant: Robert Bosch GmbH, Stuttgart (DE)

(72) Inventors: Alexander Zimmer, Stuttgart (DE); Andreas Petersen, Eningen (DE); Johannes Meyer, Haseluenne (DE); Stefan Gering, Weissach (DE); Thomas Alexander Schlebusch, Renningen (DE)

(21) Appl. No.: 18/548,281

(22) PCT Filed: Jun. 8, 2022
(86) PCT No.: PCT/EP2022/065501

§ 371 Date: Aug. 29, 2023

(30) Foreign Application Priority Data
Jul. 15, 2021 (DE) 10 2021 207 540.1

(51) Int. Cl. A61B 3/113; G01B 9/02; G01P 3/36; G06F 3/01

(52) U.S. Cl. CPC A61B 3/113; G01B 9/02092; G01P 3/36; G06F 3/013

(57) ABSTRACT

A method for determining an eye position. The method includes: receiving laser feedback interferometry measurement values of a laser feedback interferometry measurement of an eye by means of at least one laser interferometry unit; determining a velocity component of the component of the eye relative to the laser feedback interferometry unit based on the laser feedback interferometry measurement values; determining a rotational velocity of the eye about an axis of rotation based on the rotational velocity about the axis of rotation by integrating the rotational velocity over a predetermined time segment; and providing the eye position.

### FIELD

The present invention relates to a method for operating an eye position.

### BACKGROUND INFORMATION

Various systems for determining eye positions or eye tracking are described in the related art. For example, there are camera-based systems that use image or video data to track eye movements. In addition, there are systems that determine electrical properties of the retina in order to determine the eye position. Furthermore, U.S. Patent Application Publication No. US 2016/166146 A describes a system that determines an orientation of the eye based on an intensity of a light signal reflected on an eye component.

### SUMMARY

It is an object of the present invention to provide an improved method for determining an eye position.

This object may achieved by the method for determining an eye position of the present invention. Advantageous configurations of the present invention are disclosed herein.

According to one aspect of the present invention, a method for determining an eye position is provided. According to an example embodiment of the present invention, the method includes:
receiving laser feedback interferometry measurement values of a laser feedback interferometry measurement of an eye by means of at least one laser interferometry unit, wherein the measurement values are based on at least one laser signal reflected on a component of the eye;
determining a velocity component of the component of the eye relative to the laser feedback interferometry unit based on the laser feedback interferometry measurement values;
determining a rotational velocity of the eye about an axis of rotation based on the velocity component by using a geometric eye model, wherein the geometric eye model describes a function between the velocity component and the rotational velocity about the axis of rotation;
ascertaining the eye position based on the rotational velocity about the axis of rotation by integrating the rotational velocity over a predetermined time segment; and
providing the eye position.

This can achieve the technical advantage that an improved method for determining an eye position can be provided. For this purpose, according to an example embodiment of the present invention, based on measurement values of laser feedback interferometry measurements of an eye, velocity components of the eye, in particular of at least one component of the eye, on which a reflection of the laser signals of at least one laser feedback interferometry unit are reflected, are determined. Based on the determined velocity component, a corresponding rotational velocity of the eye is determined by taking into account a geometric eye model which describes a function between the velocity component and a rotational velocity about an axis of rotation of the eye. By integrating the rotational velocity over a predetermined time segment, an eye position of the eye can be ascertained and provided.

By using laser feedback interferometry measurements to determine an eye position, flexible determination of eye positions can be achieved. In particular, due to the technically simple and space-saving design of corresponding laser feedback interferometry units, these measurements can be performed in devices that only provide a limited spatial area. For example, one possibility is to provide a corresponding eye position determination for data glasses, e.g., smartglasses or augmented reality glasses, in which an eye position in the form of eye tracking must be carried out in order to be able to ensure that information to be displayed is displayed in an area of the lens that matches a viewing direction of the eye of a wearer of the data glasses.

According to an example embodiment of the present invention, by using the geometric eye model, which uses geometrical considerations of the human eye to describe the function between the measured velocity component and a rotational velocity of the eye, and by using the physiological eye movement model, which takes into account a physiological consideration of eye dynamics of the human eye, a precise determination of an eye position based on the measured velocity components of the laser feedback interferometry measurement can be provided. As a result, an improved determination of an eye position based on laser feedback interferometry measurements can be achieved.

According to an example embodiment of the present invention, by integrating the rotational velocity over a predetermined time segment, a precise eye position can be determined. The eye position can in this case be expressed in the form of a rotation angle relative to the respective axis of rotation. The rotational velocity accordingly is a temporal change of the rotation angle caused by the rotational movement of the eye about the respective axis of rotation. Based on the ascertained rotational velocity, a corresponding endpoint of a rotational movement of the eye about the axis of rotation according to the ascertained rotational velocity can thus be defined, by integration, as an eye position, which the eye reaches after the predetermined time segment has elapsed. In the sense of the application, the eye position in this case corresponds to a viewing direction of the eye. By integration, a very precise determination of the eye position or viewing direction of up to 1° degree accuracy can be achieved. In addition, the integration enables a technically simple and computationally efficient method for determining the eye position.

A velocity component in the sense of the present application is to be understood as a vector component of a velocity vector.

An eye component in the sense of the present application is a physical component of the eye and may comprise a cornea, a lens, an iris, a retina, a sclera or other components.

According to one example embodiment of the present invention, laser feedback interferometry measurement values of laser feedback interferometry measurements by at least two laser interferometry units are received, wherein velocity components are determined for the measurement values of the at least two laser interferometry units, and wherein the method furthermore comprises:
determining distance components of the component of the eye relative to the laser feedback interferometry units based on the laser feedback interferometry measurement values of the laser feedback interferometry units;
determining an eye position based on the distance components by performing a triangulation calculation by taking into account the relative positions between the at least two laser feedback interferometry units and the eye; and
using the determined eye position as a starting point in the integration of the velocity components.

This can achieve the technical advantage that an improved determination of the eye position can be achieved. For this purpose, an eye position is ascertained based on the determined two distance components by taking into account triangulation ratios between the at least two laser feedback interferometry units and the eye to be measured. By using the thus determined starting point, the eye position ascertained by integration can be specified more precisely.

A distance component in the sense of the present application is to be understood as a vector component of a distance vector.

According to one example embodiment of the present invention, the method furthermore comprises:
determining a signal-to-noise ratio of the measurement values of the laser feedback interferometry unit;
ascertaining an abrupt change in the signal-to-noise ratio for temporally successively recorded measurement values of the laser feedback interferometry unit;
identifying the temporally consecutive measurement values as measurement values of laser signals reflected by at least two different eye components, by taking into account the abrupt change in the signal-to-noise ratio of the temporally consecutive measurement values;
identifying a transition between the at least two eye components; and
determining an eye position based on the identified transition between at least two eye components.

This can achieve the technical advantage that an alternative approach for determining the eye position can be provided. When ascertaining an abrupt change in the signal-to-noise ratio of temporally successively recorded measurement values, a transition between at least two eye components is detected due to the abrupt change and different reflectivities of different eye components. By taking into account the physiological configuration of the eye and the relative arrangement between the different eye components, a precise eye position can thus be ascertained based on the ascertained transition between two eye components.

According to one example embodiment of the present invention, the method furthermore comprises:
identifying the eye component based on the signal-to-noise ratio by taking into account individual reflectivities of the eye components of the eye; and/or
identifying the eye components based on the distance components by taking into account a physiological structure of the eye;
wherein the eye components are identified as an iris, a retina, or a sclera of the eye.

This can achieve the technical advantage that precise identification of the eye components relevant to the determination of the eye position is enabled.

According to one example embodiment of the present invention, the method furthermore comprises:
predicting a saccadic endpoint of a movement of the eye based on the determined eye position by taking into account a physiological description of a saccadic eye movement of an eye; and
providing the predicted saccadic endpoint as an eye position predicted for a predetermined time point.

This can achieve the technical advantage that an additional predicted eye position in the form of a predicted saccadic endpoint can be provided by taking into account saccadic eye movements. This can achieve or increase a more precise specification or a scope of the provided information content of the method according to the present invention in the form of the additional predicted eye position.

According to one example embodiment of the present invention, the method furthermore comprises:
performing a correction of the eye position ascertained by integrating the rotational velocity in the method step, by taking into account the eye position ascertained by the triangulation calculations carried out in the method step and/or the eye position ascertained in the method step by means of the abrupt change in the signal-to-noise ratio and/or the eye position ascertained in the method step by means of the saccadic endpoint prediction.

This can achieve the technical advantage that a more precise specification of the determination of the eye position is enabled.

According to one example embodiment of the present invention, the velocity component of a tangential velocity component is defined within a reflection plane of the laser signal defined by the respective component of the eye, wherein the tangential velocity component is parallel to a direction of the laser signal.

This can achieve the technical advantage that a model simplification can be achieved in that the velocity components to be taken into account can be limited to a tangential velocity component. This decreases a complexity of the calculations to be carried out and, associated therewith, a computing capacity or duration required for carrying out the method according to the present invention.

According to one example embodiment of the present invention, the geometric eye model comprises a first velocity model, a second velocity model and a third velocity model, wherein the first velocity model describes a function between the velocity component and the rotational velocity about the axis of rotation for a laser signal reflected on the retina, wherein the second velocity model describes a function between the velocity component and the rotational velocity about the axis of rotation for a laser signal reflected on the iris, and wherein the third velocity model describes a function between the velocity component and the rotational velocity about the axis of rotation for a laser signal reflected on the sclera.

This can achieve the technical advantage that a precise determination of the eye position based on the geometric eye model is enabled in that, as a function of the point of impingement of the laser signals on the respectively defined components of the eye, the properties of the respective eye component can be taken into account in that corresponding velocity models for the respective eye components retina, iris, sclera are used as submodels of the geometric eye model for determining the rotational velocity.

According to one example embodiment of the present invention, a shape of the retina is approximated with a spherically shaped surface in the first velocity model, wherein a shape of the iris is approximated with a planarly shaped surface in the second velocity model, and wherein a shape of the sclera is approximated with a spherically shaped surface in the third velocity model.

This can achieve the technical advantage that geometrically simple approximations of the surface configurations of the individual eye components retina, iris, sclera enable a further simplification of the first to third velocity models and, associated therewith, a further acceleration of the method according to the present invention, including a reduction of the required computing capacity.

According to one example embodiment, the following applies to the first velocity model and the third velocity model: V_{T}=(I×q·ω)·l, wherein I is a vector representation of a point of impingement of the laser signal on the eye component, q is a vector representation of an axis of rotation of the eye, l is a vector representation of the laser signal and ω is an angular velocity about the axis of rotation, and wherein the following applies to the second velocity model:

$${V}_{T} = \frac{Δ d}{dt} + \frac{{v}_{Ts 1} + {v}_{Ts 2}}{2} ,$$

wherein Δd is a distance increment on the iris and v_{Ts1},v_{Ts2} are two transverse velocity components ascertained at time points t1, t2.

This can achieve the technical advantage that a precise determination of the rotational velocity is enabled by taking into account the geometrical properties of the eye components retina, iris, sclera.

According to one example embodiment of the present invention, the geometric eye model comprises a first distance model, a second distance model and a third distance model, wherein the first distance model describes a distance between the laser feedback interferometry unit and the retina, wherein the second distance model describes a distance between the laser feedback interferometry unit and the iris, wherein the third distance model describes a distance between the laser feedback interferometry unit and the sclera, wherein the following applies to the first distance model: d=dₗₛ(r_{Retina}), where d is the distance component, S is a position of the laser feedback interferometry unit, l is the laser signal and r_{Retina} is a radial distance between the retina and an eye center, wherein the following applies to the third distance model: d=dₗₛ(r_{Sclera}), where r_{Retina} is a radial distance between the sclera and the eye center, and wherein the following applies to the second distance model:

$$d = \frac{( {R}_{p} {e}_{0} - S ) \cdot ( {R}_{p} e )}{l \cdot ( {R}_{p} e )} ,$$

wherein Rₚ is a rotation matrix of the iris, e₀ is a unit vector from the eye center to the iris, e is a unit vector of an iris surface and l is a vector representation of the laser signal.

This can achieve the technical advantage that a precise identification of the eye component as retina, iris, sclera based on the distance model is enabled in that corresponding distance components can be determined according to the geometric model by taking into account the geometrical properties of the respective eye components. In this case, the distance models are simplified based on the geometrical properties of the geometric model so that a precise distance determination that is at the same time simple to carry out and efficient in terms of computing power is enabled.

According to one example embodiment of the present invention, the ascertained eye position is a future eye position that the eye will assume at a predetermined future time point based on the determined rotational velocity.

This can achieve the technical advantage that both a current eye position present at the time point of the interferometry measurement and a future eye position that will be assumed after the completion of the present eye movement can be determined.

According to one example embodiment of the present invention, the at least two laser interferometry units are arranged relative to the eye in such a way that eye rotational movements about at least two axes of rotation arranged perpendicularly to one another can be determined, and wherein rotational movements of the eye about the at least two axes of rotation arranged perpendicularly to one another can describe every physiologically possible eye position of a human eye.

This can achieve the technical advantage that a complete eye movement of the human eye about two axes of rotation arranged perpendicularly to one another can be taken into account and a precise determination of the eye position is thus enabled.

According to a second aspect of the present invention, a computing unit is provided, wherein the computing unit is configured to perform the method for determining an eye position according to one of the above-described embodiments.

According to a third aspect of the present invention, a computer program product is provided, comprising instructions that, when the program is executed by a data processing unit, cause the latter to perform the method for determining an eye position according to one of the above-described embodiments.

Exemplary embodiments of the present invention are explained with reference to the figures.

### BRIEF DESCRIPTION OF THE DRAWINGS

FIG. 1 shows a schematic representation of a system for determining an eye position according to one example embodiment of the present invention.

FIG. 2 shows a schematic representation of geometric components of a determination of an eye position, according to an example embodiment of the present invention.

FIGS. 3A-3C show a schematic representation of a geometric model for determining an eye position, according to an example embodiment of the present invention.

FIG. 4 shows a flow chart of a method for determining an eye position according to one example embodiment of the present invention.

FIG. 5 shows a further schematic representation of the system for determining an eye position according to one example embodiment of the present invention.

FIG. 6 shows a schematic representation of a computer program product.

### DETAILED DESCRIPTION OF EXAMPLE EMBODIMENTS

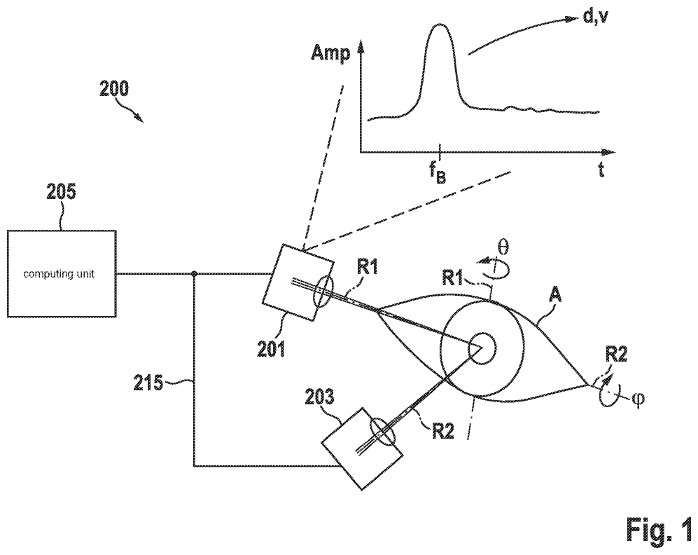

FIG. 1 shows a schematic representation of a system 200 for determining an eye position according to one embodiment.

In the embodiment shown, the system 200 for determining an eye position comprises a first laser feedback interferometry unit 201 and a second laser feedback interferometry unit 203, which are connected to a computing unit 205 via a data link 215.

The first and second laser feedback interferometry units 201, 203 are designed to carry out laser feedback interferometry measurements and, for this purpose, to emit frequency-modulated laser signals L1, L2, to receive laser signals reflected on an object, and to provide distance components d and/or velocity components v of a respective object via laser feedback interferometry, laser self-mixing, and the determination of beat frequencies f_{B}.

In the embodiment shown, the first and second laser feedback interferometry units 201, 203 are arranged relative to an eye A in such a way that the respective laser feedback interferometry units 201, 203 can ascertain eye movements of the eye A by recording corresponding velocity components with respect to two axes of rotation R1, R2 arranged perpendicularly to one another. By such an arrangement, any eye movement of the eye A can be represented as a corresponding linear combination of rotational movements about the first and second axes of rotation R1, R2 or can be ascertained by the respective laser feedback interferometry units 201, 203. A corresponding eye position of the eye A in the representation shown can be represented by specifying the respective rotation angles θ and φ.

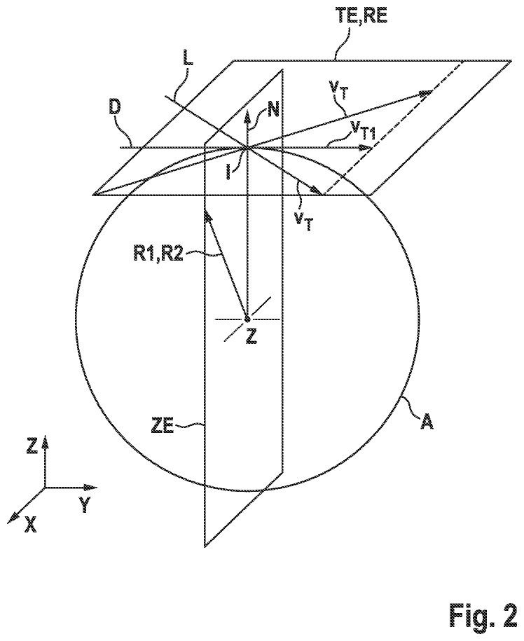

FIG. 2 shows a schematic representation of geometric components of a determination of an eye position.

FIG. 2 shows that the velocity component v accessible by interferometry measurements can be reduced to a tangential velocity v_{T}, v_{T1} within a reflection plane RE of the eye component resulting in the reflection of the laser signal L. In the embodiment shown, the shape of the eye A is approximated as an evenly formed sphere. In the consideration shown, the irradiation of a laser signal L of a laser feedback interferometry unit 201, 203 results in a reflection of the laser signal L on the spherical surface of the eye A. The laser feedback interferometry measurement is limited with respect to the velocity component v to a velocity component of the respectively detected object that is parallel to a direction of the laser signal L with respect to a point of impingement I of the laser signal L on the respective object to be detected.

Assuming that eye movements are limited to rotational movements about an axis of rotation R passing through a center Z of the eye A, the velocity components of the movement of the eye A, which are accessible through the velocity component v of a laser feedback interferometry measurement carried out according to the shown laser signal L, are limited to the shown tangential velocities v_{T}, v_{T1} which are arranged within the tangential plane TE or the reflection plane RE, on which the respective laser signal L is reflected. Since the laser interferometry measurement in the form of the laser signal L shown is exclusively sensitive to the velocity components parallel to the direction of the laser signal L, only the tangential velocity v_{T1} parallel to the shown direction D of the laser signal L is accessible through the respective velocity component v of the laser signal L shown. The tangential velocity v_{T1} can in this case be interpreted as a projection of the shown tangential velocities v_{T} onto the direction of the laser signal L. Due to the previously carried-out limitation of the movement of the eye A, the direction of the laser signal L is limited to the direction D arranged within the tangential plane TE.

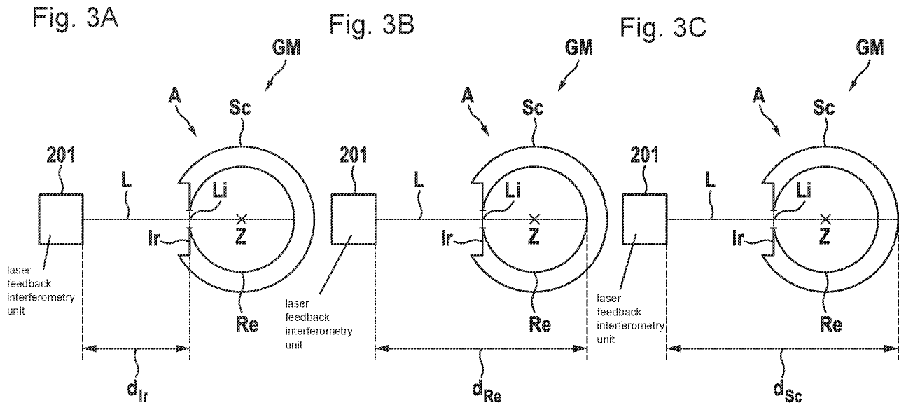

FIGS. 3A-3C show a schematic representation of a geometric model for determining an eye position.

FIGS. 3A-3C show a geometric model consideration of the eye A, which is taken into account for the determination of the rotational velocity based on the distance or velocity components d, v measured in the laser feedback interferometry measurement. The geometric model GM shown here comprises a geometrically simplified description of the eye A and, in particular, of the eye components retina Re, iris Ir, and sclera Sc which are relevant to the eye position determination. The geometric model GM provides that the eye components retina Re and sclera Sc are approximately taken into account with a spherical surface. In contrast, the geometric model GM provides that the iris Ir is approximately taken into account in the form of a planar surface. In the simplest form, the geometric model GM provides that both the spherical surfaces of the retina Re and sclera Sc and the planar surface of the iris Ir are taken into account as optimally spherical or optimally planar surfaces, wherein the spherical surfaces of the retina Re and sclera Sc have a uniform and constant curvature at every point of the surface, while the planar surface of the iris Ir is completely planar.

The geometric model GM furthermore provides that the spherical surfaces of the retina Re and sclera Sc are arranged concentrically around a center Z of the eye A. The planar surface of the iris Ir is arranged in a tangential orientation to the spherical surface of the retina Re or to the spherical surface of the sclera Sc and has an opening, by means of which a lens Li of the eye A is taken into account, in a central position. FIGS. 3A-3C show only a two-dimensional geometric model of the eye A, which shows a sectional view of the eye A through a planar sectional plane through the center Z and through a midpoint of the iris Ir. Alternatively, the geometric model GM may be designed as a three-dimensional geometric model of the eye. In the three-dimensional geometric model GM, the eye components retina Re and sclera Sc are thus formed as concentrically arranged three-dimensional spherical bodies, while the eye component iris Ir is represented as a planar circular surface with a circular opening which is arranged in a center of the surface and by means of which the lens Li is taken into account.

The geometric model GM shown is reflected in the below-mentioned Equations 1 to 6 of the distance model or the velocity model, by means of which corresponding distances of the individual eye components retina Re, iris Ir, sclera Sc can be ascertained based on the distance components d or velocity components v of the laser feedback interferometry measurements by taking into account the respective geometric configurations.

In addition to the shown geometric configurations of the respective surfaces of the eye components, the geometric model GM may additionally comprise corresponding orientations or distance ratios of the individual surfaces of the eye components to one another. In addition, the geometric model GM may take into account reflection behaviors of the individual surfaces of the eye components shown, so that, in addition to the geometrical considerations of the individual eye components, additional physiological properties can be taken into account. The reflection properties of the individual eye components can be taken into account, for example, in a signal-to-noise ratio of the measurement values of the laser feedback interferometry measurements so that, based on the signal-to-noise ratios, which result in a correspondingly good signal-to-noise ratio for eye components with increased reflectivity, are enabled for the identification of the individual eye components based on the measurement values of the laser feedback interferometry measurements.

In FIGS. 3A-3C, three measurement scenarios are shown. FIG. 3A shows a measurement situation in which a laser signal L is reflected by the surface of the iris Ir. FIG. 3B shows a laser signal L, which passes through the lens opening of the lens Li and is reflected by the spherical surface of the retina Re. FIG. 3C shows a reflection on the surface of the sclera Sc.

Due to the different path lengths of the laser signals L for the three different measurement situations, with an unchanged distance between the laser feedback interferometry unit 201 and the center Z of the eye A, the three measurement situations shown result in different distance components d for the different eye components iris Ir, retina Re and sclera.

Via the distance components d, which can respectively be taken from the measurement values of the laser feedback interferometry measurements, the distances d_{Ir} of the iris Ir, d_{Re} of the retina Re and d_{Sc} of the sclera for the three measurement situations shown can be determined as the respective distances between said eye components and the laser feedback interferometry unit 201.

Via the respective determinations of the distance components d or the distances d_{Ir}, d_{Er}, d_{Sc}, the three measurement situations shown can thus be distinguished from one another so that, based on the measured distance components d, a statement can be made as to whether the respective laser signal L has impinged on, or has been reflected by, the iris, the retina or the sclera.

Alternatively or additionally, in the form of the signal-to-noise ratios of the respective measurement signals of the laser feedback interferometry measurements, the reflectivities of the individual eye components can be taken into account for the identification. Iris, retina and sclera each have different reflectivities resulting in characteristic signal-to-noise ratios.

The following Equations (1), (2), (3) describe distance models which are based on the geometric model GM shown and enable, based on the geometrical properties of the eye components defined in the geometric model GM, distance descriptions of the different eye components iris, retina, and sclera based on the respectively measured distance components d for different eye positions and/or viewing direction of the eye A with respect to a laser feedback interferometry unit 201. According to the distance models, the following applies:

$$d = \{ \begin{matrix}{d}_{Re} ( {r}_{Retina} ) & ( 1 ) \\ \frac{( {R}_{p} \cdot {e}_{0} - S ) \cdot ( {R}_{p} \cdot e )}{L \cdot ( {R}_{p} \cdot e )} & ( 2 ) \\ {d}_{Sc} ( {r}_{Sclera} ) & ( 3 )\end{matrix}$$

Equation (1) applies to a reflection on the retina Re, Equation (2) applies to a reflection on the iris Ir, and Equation (3) applies to a reflection on the sclera Sc.

In the equations, d are the distance components of the laser feedback interferometry measurements and describe the distance between the reflection surface of the eye A and the laser feedback interferometry unit 201.

The parameters r_{Retina} and r_{Sclera} respectively describe a distance of the respective eye component from the center Z of the eye. Since the surface of the retina Re and the surface of sclera Sc are approximated as spherical surfaces in the geometric model GM, Equations (1) and (3) express a directional independence of the distance of the respective eye component from the laser feedback interferometry unit 201 so that the respective distance is independent of an impingement angle of the laser signal L on the respective surface of the retina Re or sclera Sc.

In Equation (2), Rₚ, a rotation matrix of the iris Ir approximated as a planar surface, is taken into account. The parameter S describes a position of the laser feedback interferometry unit 201 relative to the eye A. The parameter L describes a vector representation of the laser signal impinging on the iris Ir. The parameter e₀ describes a unit vector from the center Z of the eye A to the surface of the iris Ir, while the parameter e describes a unit vector within the surface of the iris Ir.

As described above, in the geometric model GM, the surface of the iris is approximated as a planar surface. This has the result that a distance of the iris Ir from the laser feedback interferometry unit 201 changes as a function of the eye position and thus as a function of an orientation of the surface of the iris Ir to the laser feedback interferometry unit 201. This effect is taken into account by the rotation matrix Rₚ.

The following Equations (4), (5), (6) describe velocity models which are based on the geometric model GM and by means of which corresponding rotational velocities of the eye A can be derived for the above-described measurement situations based on the respectively measured velocity components v.

Following the statements regarding FIG. 2, due to the limited eye movement, these velocity components can be reduced to corresponding tangential velocities v_{T} which are arranged within a reflection plane RE of the laser signal L and oriented parallel to the projection of the direction D of the laser signal L onto this reflection plane RE. By taking into account the geometrical properties of the eye components iris, retina, sclera defined in the geometric model GM, the following equations express corresponding dependencies of the measured tangential velocities on actual rotational velocities about corresponding axes of rotation R1, R2 of the eye A. Via the velocity models, determinations of the rotational velocities of the eye movements based on the velocity components v of the laser feedback interferometry measurements are thus enabled. According to the velocity models, the following applies:

$$v = {v}_{T} = \{ \begin{matrix}( I \times R \cdot ω ) \cdot L & ( 4 ) \\ \frac{Δ d}{dt} + \frac{{v}_{Ts 1} + {v}_{Ts 2}}{2} & ( 5 ) \\ ( I \times R \cdot ω ) \cdot L & ( 6 )\end{matrix}$$

Analogously to the above-mentioned distance model, Equation (4) is applicable in the shown velocity model when the laser signal impinges on the retina Re. Equation (5) is applicable when the laser signal impinges on the iris Ir. Equation (6) is applicable when the laser signal impinges on the sclera Sc of the eye.

The parameter v in the equations shown is the velocity component which can be ascertained from the measurement values of the laser feedback interferometry unit 201 and describes a velocity of the eye A or of the respective component of the eye A relative to the laser feedback interferometry unit 201. As described above, according to the geometrical considerations mentioned, this velocity component can be reduced to a tangential velocity v_{T} which is arranged in a corresponding tangential plane TE or reflection plane RE defined by the respective eye component on which the reflection of the laser signal L occurs.

The tangential velocity v_{T}, which can be ascertained through the laser signal L, is in particular defined by the component arranged parallel to the direction D of the laser signal L. This fact is expressed in Equations (4) and (6) by the vector product I×R between the vector representation of the point of impingement I of the laser signal L on the respective eye component and the vector representation of the axis of rotation R and the projection of the resulting vector onto the vector representation of the laser signal L. The parameter ω describes a rotational velocity of the eye A about the respective axis of rotation R1, R2. An example of vector representations I and R is shown in FIG. 2.

Based on the measured velocity components v, a corresponding rotational velocity ω of the eye about a corresponding axis of rotation R1, R2 can thus be determined by backward evaluation of Equations (4) and (6). The respective axis of rotation R1, R2 can be defined by the respective arrangement or orientation of the laser feedback interferometry unit 201 relative to the eye A.

Equation (5) shows a relationship between the measured velocity component v and the change in the distance of the iris Ir, approximated as a planar surface, from the laser feedback interferometry unit 201. Rotation of the eye A causes tilting of the iris Ir relative to the laser feedback interferometry unit 201, which ensures that the change in distance between the iris Ir and the laser feedback interferometry unit 201 is ascertained. By taking into account the distance term of Equation (2) and the rotation matrix Rₚ defined therein and by a corresponding temporal rotation of the iris Ir and an associated distance change Δd/dt according to Equation (2), a rotational velocity can be ascertained. For this purpose, tangential velocities v_{Ts1,s2} at two different time points t1, t2 are taken into account in Equation (5).

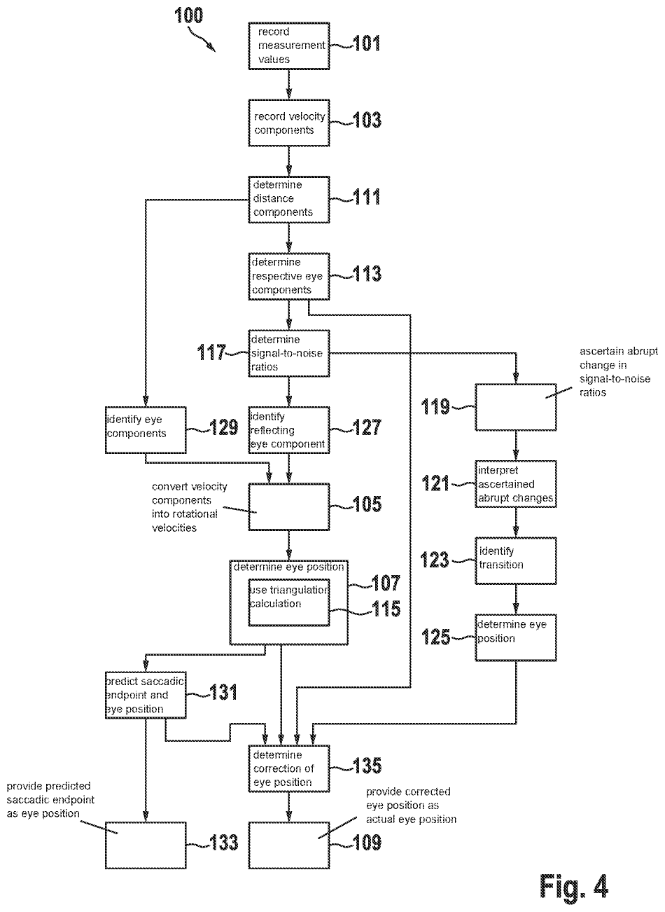

FIG. 4 shows a flow chart of a method 100 for determining an eye position according to one embodiment.

In order to determine an eye position according to the method 100 according to the present invention, measurement values from the feedback interferometry measurements are first recorded in a method step 101 by at least two laser feedback interferometry units 201, 203, wherein the measurement values are based on laser signals L, which are emitted by the laser feedback interferometry units 201, 203 in the direction of the eye A to be examined, are reflected back by an eye component of the eye A in the direction of the respective laser feedback interferometry unit 201, 203 and are received by the latter.

In a method step 103, velocity components v1, v2 are recorded based on the measurement values of the laser feedback interferometry measurements. The velocity components v1, v2 respectively correspond to the components of the velocity of an eye movement relative to the respective laser feedback interferometry units 201, 203. According to the geometrical considerations mentioned with respect to FIG. 2, the velocity components v1, v2 can be reduced to corresponding tangential velocities v_{T1}, v_{T2} arranged within a corresponding reflection plane RE and oriented parallel to a projection of a direction d of a respective laser signal L1, L2 in the respective reflection plane Re.

In a method step 111, distance components d1, d2 of the reflecting eye component are furthermore determined based on the measurement values of the laser feedback interferometry measurements of the individual laser feedback interferometry units 201, 203.

In a method step 129, the respective eye components can furthermore be identified based on the distance components d1, d2 determined in method step 111 and by taking into account the arrangements of the individual eye components within the eye A.

In a method step 113, an eye position of the eye A is ascertained based on the ascertained distance components d1, d2 and by performing a triangulation calculation by taking into account the relative position between the at least two laser feedback interferometry units 201, 203 and the eye A to be examined. The eye position of the eye A comprises a viewing direction of the eye A relative to the at least two laser feedback interferometry units 201, 203. By taking into account the distance components d1, d2, which respectively describe the relative distances between the respective laser feedback interferometry units 201, 203 and the respective eye components, and a relative distance between the at least two laser feedback interferometry units 201, 203 and by taking into account a physiological eye model, by means of which an unambiguous arrangement of the individual eye components within the eye is enabled, a viewing direction of the eye and an associated eye position can thus be determined.

In a further method step 117, corresponding signal-to-noise ratios SNR1, SNR2, which express a ratio between the signal of the beat frequency f_{B} and the signal background of the respective frequency spectrum, are furthermore determined based on the laser feedback interferometry measurement values of the individual laser feedback interferometry units 201, 203.

In a method step 127, the reflecting eye component is identified based on the distance components d1, d2 by taking into account the physiological eye model. Alternatively or additionally, the respective eye component is identified by taking into account the different signal-to-noise ratios SNR1, SNR2 and by taking into account the physiological eye model in which the reflectivities of the individual eye components are expressed. Eye components with different reflectivities cause different signal intensities and correspondingly different signal-to-noise ratios. The eye components are in particular identified as iris Ir, retina Re or sclera Sc of the eye A.

In a method step 105, the velocity components v1, v2 determined in method step 103 are subsequently converted into corresponding rotational velocities about respective axes of rotation R1, R2 by taking into account the geometric model GM described above. The above-described Equations 1, 2, 3 of the distance model as well as Equations 4, 5, 6 of the velocity model can be used for this purpose. In particular, based on the eye components identified in method step 127, the respective equations of the distance or velocity model can be selected in measurement situations described above, so that Equations 1 and 4 are selected if the eye component is identified as the retina Re, while Equations 2 and 5 are selected if the eye component is identified as the iris Ir, and Equations 3 and 6 are selected if the eye component is identified as sclera Sc.

In a method step 107, by taking into account the calculated rotational velocities, a corresponding eye position is determined by performing an integration of the rotational velocities over a predetermined time segment. For this purpose, in a method step 115, the eye position determined in method step 113 by means of the triangulation calculation is used as the start value for the integration of the rotational velocities. By using the ascertained eye position, which can be expressed as an angle pair of the rotation angles θ and φ about the respective axes of rotation R1, R2, as the starting point for the time integration of the rotational velocities, which can respectively be expressed as temporal changes of the two rotation angles θ and φ, an eye position, which the eye will assume according to the starting point and the rotational velocities after the predetermined time segment, can thus be determined in the form of changed rotation angles θ and φ. The predetermined time segment can in this case be configured in such a way that, if the laser feedback interferometry measurement values are recorded cyclically and if the method 100 according to the present invention is carried out cyclically, the time segment is in each case adjusted to the cycle times of the individual laser feedback interferometry measurements.

In a method step 119, an abrupt change in the signal-to-noise ratios of temporally consecutive measurement values of temporally consecutive laser feedback interferometry measurements can furthermore be ascertained based on the signal-to-noise ratios determined in method step 117.

In a method step 121, the ascertained abrupt changes in the signal-to-noise ratios can be interpreted in that the laser signals L1, L2 of the temporally successive measurement values were reflected by different eye components of the eye A so that a transition of the laser signals from one eye component to another eye component occurred due to an eye movement that was performed. Due to the different reflectivities of the different eye components, a corresponding abrupt change in the signal-to-noise ratios can occur as a result.

In a method step 123, the corresponding transition between the different eye components resulting in the reflection of the laser signals L1, L2 is identified. For example, as a result of a performed eye movement, a laser signal that previously impinged on the iris and was reflected by the iris may now impinge through the lens on the retina and be reflected by the retina. Such a transition can be ascertained precisely through a substantial change in the signal-to-noise ratio.

In a method step 125, based on the ascertained transition between the different eye components, a corresponding eye position of the eye A can be determined by taking into account the physiological eye model, in which the respective reflectivities of the individual eye components are taken into account, and by taking into account the arrangement of the individual eye components within eye A relative to one another.

In a method step 131, a prediction of a saccadic endpoint as the endpoint of a saccadic eye movement and a corresponding eye position can furthermore be predicted by using a physiological model for describing a saccadic eye movement.

In a method step 135, a correction of the eye position determined in method step 107 can be carried out by taking into account the eye position determined in method step 125 based on the transition between the at least two eye components and/or by taking into account the eye position determined in method step 113 based on the triangulation consideration and/or by taking into account the eye position determined in method step 131 in the form of the predicted saccadic endpoint. The correction of the eye position can in this case include that the eye position determined in method step 107 is adjusted accordingly. Alternatively, for example, the eye position ascertained in method step 125 based on the transition between the at least two eye components may be determined as the actual eye position and the eye position determined in method step 107 based on the integration of the rotational velocities may be discarded.

In a method step 109, the corrected eye position generated in method step 135 can be provided as the actual eye position. As already mentioned above, the eye position can be described as an angle pair of two rotation angles θ, φ about the respectively defined axes of rotation, R1, R2, which are preferably arranged perpendicularly to one another, and can comprise a viewing direction of the eye relative to the respective laser feedback interferometry units 201, 203.

Alternatively or additionally, in a method step 133, the eye position determined in method step 131 in the form of the predicted saccadic endpoint may be provided as the eye position.

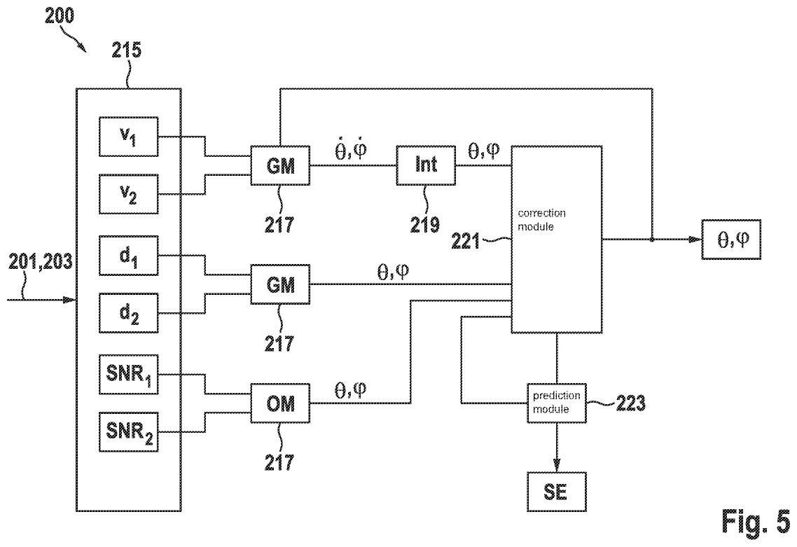

FIG. 5 shows a further schematic representation of a system 200 for determining an eye position according to one embodiment.

The measurement values of the laser feedback interferometry measurements of the at least two laser feedback interferometry units 201, 203 are first received by an input module 215. Based on the measurement values, two velocity components v1, v2, two distance components d1, d2 and two signal-to-noise ratios SNR1, SNR2 are respectively subsequently generated. The velocity components v1, v2, distance components d1, d2 and signal-to-noise ratios SNR1, SNR2 are respectively based on the different measurement values of the at least two laser feedback interferometry units 201, 203.

By performing the geometric model GM described above in an application module 217, a corresponding eye position in the form of the angle pair θ, φ of the rotation angles about the respective axes of rotation R1, R2 can be determined based on the two distance components d1, d2 and by taking into account or performing the above-described triangulation considerations. Furthermore, by performing the geometric model in the application module 217, corresponding rotational velocities \dot{θ}, \dot{φ} can be ascertained as temporal changes in the rotation angles θ and φ based on the two velocity components v1, v2.

In an integration module 219, integration of the rotational velocities \dot{θ}, \dot{φ} can be carried out thereafter and a corresponding eye position in the form of the angle pair θ, φ can be generated based thereon. For this purpose, the eye position previously determined by taking into account the triangulation consideration based on the two distance components d1, d2 can be used as a corresponding starting point of the integration of the integration module 219.

Alternatively, by performing an optical eye model OM, which takes into account the physiological properties of the individual eye components and in particular the reflectivities of the different eye components, a corresponding eye position in the form of the angle pair θ, φ can be generated based on the signal-to-noise ratios SNR1, SNR2 when an abrupt change in the signal-to-noise ratio SNR1, SNR2 and a transition between two eye components causing it are detected. However, such a transition between two eye components does not occur during every eye movement of the eye A relative to the laser feedback interferometry units 201, 203 so that the determination of the eye position based on the change in the signal-to-noise ratio takes place only in addition to the above-described determination of the eye position based on the integration of the rotational velocities.

As described above, the method 100 according to the present invention can be performed cyclically so that, if the laser feedback interferometry measurement and the corresponding provision of the laser feedback interferometry measurement values are performed cyclically, corresponding rotational velocities or eye positions are generated. Via a correction module 221, a continuous correction of the eye position ascertained by integration of the determined rotational velocities \dot{θ}, \dot{φ} in the integration module 219 can be carried out by taking into account the eye position ascertained via the triangulation consideration or by taking into account the eye position ascertained via the detection of the abrupt change in the signal-to-noise ratio, so that, after each completion of a measurement cycle and after each determination of a corresponding eye position, a corresponding correction of the eye position can be carried out by the correction module 221.

For this purpose, a corresponding prediction of a saccadic endpoint of a saccadic eye movement can furthermore be generated via a prediction module 223, can in turn serve as an input into the correction module 221 and can thus be used to correct the eye position generated by integrating the rotational velocity.

After correction of the eye position by the correction module 221, a corresponding corrected eye position in the form of the angle pair θ, φ can be provided. As described above, in order to correct the eye position, the eye position generated via the integration of the rotational velocities can be adjusted accordingly or the eye position ascertained via the abrupt change in the signal-to-noise ratios and the transition detected as a result between at least two eye components can in this case be provided as the actual eye position.

Furthermore, additionally or alternatively, the predicted saccadic endpoint may be provided as a further eye position.

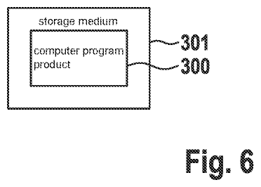

FIG. 6 shows a schematic representation of a computer program product 300 according to one embodiment, wherein the computer program product 300 comprises instructions that, when the program is executed by a data processing unit, cause the latter to perform the method 100 for determining an eye position according to one of the embodiments described above.

In the embodiment shown, the computer program product 300 is stored in a storage medium 301. The storage medium 301 may be a commercially available storage medium.

1-15. (canceled)

16. A method for determining an eye position, comprising the following steps:
receiving laser feedback interferometry measurement values of a laser feedback interferometry measurement of an eye using at least one laser interferometry unit, wherein the laser feedback interferometry measurement values are based on at least one laser signal reflected on a component of the eye;
determining a velocity component of the component of the eye relative to the laser feedback interferometry unit based on the laser feedback interferometry measurement values;
determining a rotational velocity of the eye about an axis of rotation based on the velocity component by using a geometric eye model, wherein the geometric eye model describes a function between the velocity component and the rotational velocity about the axis of rotation;
ascertaining an eye position based on the rotational velocity about the axis of rotation by integrating the rotational velocity over a predetermined time segment by taking into account a known eye position as a starting point of the integration; and
providing the eye position change.

17. The method according to claim 16, wherein laser feedback interferometry measurement values of laser feedback interferometry measurements using least two laser interferometry units are received, wherein velocity components are determined for the laser feedback interferometry measurement values of the at least two laser interferometry units, and wherein the method further comprises:
determining distance components of the component of the eye relative to the at least two laser feedback interferometry units based on the laser feedback interferometry measurement values of the at least two laser feedback interferometry units;
determining an eye position based on the distance components by performing a triangulation calculation by taking into account relative positions between the at least two laser feedback interferometry units and the ascertained distance components from the eye; and
using the determined eye position as the starting point in the integration of the rotational velocity.

18. The method according to claim 17, further comprising:
determining a signal-to-noise ratio of the laser feedback interferometry measurement values of the laser feedback interferometry unit;
ascertaining an abrupt change in the signal-to-noise ratio for temporally successively recorded laser feedback interferometry measurement values of the laser feedback interferometry unit;
identifying the temporally consecutive measurement values as measurement values of laser signals reflected by at least two different eye components, by taking into account the abrupt change in the signal-to-noise ratio of the temporally consecutive laser feedback interferometry measurement values;
identifying a transition between the at least two different eye components; and
determining an eye position based on the identified transition between the at least two eye components.

19. The method according to claim 18, further comprising:
identifying each eye component based on the signal-to-noise ratio by taking into account individual reflectivities of the eye components of the eye; and/or
identifying each eye component based on the distance components by taking into account a physiological structure of the eye;
wherein the eye components are identified as an iris of the eye, or a retina of the eye, or a sclera of the eye.

20. The method according to claim 19, further comprising:
predicting a saccadic endpoint of a movement of the eye based on the determined eye position by taking into account a physiological description of a saccadic eye movement of an eye; and
providing the predicted saccadic endpoint as an eye position predicted for a predetermined time point.

21. The method according to claim 20, further comprising:
performing a correction of the eye position ascertained by integrating the rotational velocity, by taking into account the eye position ascertained by the triangulation calculations and/or the eye position ascertained using the abrupt change in the signal-to-noise ratio and/or the eye position ascertained using the saccadic endpoint prediction.

22. The method according to claim 16, wherein the velocity component of a tangential velocity component is defined within a reflection plane of the laser signal defined by the respective component of the eye, and wherein the tangential velocity component is parallel to a direction of the laser signal.

23. The method according to claim 16, wherein the geometric eye model includes a first velocity model, a second velocity model, and a third velocity model, wherein the first velocity model describes a function between the velocity component and the rotational velocity about the axis of rotation for a laser signal reflected on a retina of the eye, wherein the second velocity model describes a function between the velocity component and the rotational velocity about the axis of rotation for a laser signal reflected on an iris of the eye, and wherein the third velocity model describes a function between the velocity component and the rotational velocity about the axis of rotation for a laser signal reflected on a sclera of the eye.

24. The method according to claim 23, wherein a shape of the retina is approximated with a spherically shaped surface in the first velocity model, wherein a shape of the iris is approximated with a planarly shaped surface in the second velocity model, and wherein a shape of the sclera is approximated with a spherically shaped surface in the third velocity model.

25. The method according to claim 23, wherein the following applies to the first velocity model and the third velocity model: v_{T1,2}=(I×R·ω)·L, wherein I is a vector representation of a point of impingement of the laser signal on the eye component, R is a vector representation of an axis of rotation of the eye, L is a vector representation of the laser signal and ω is an angular velocity about the axis of rotation, and wherein the following applies to the second velocity model:
$${v}_{T 1 , 2} = \frac{Δ d}{dt} + \frac{{v}_{Ts 1} + {v}_{Ts 2}}{2} ,$$
wherein Δd is a distance increment on the iris and v_{Ts1},v_{Ts2} are two tangential velocity components ascertained at time points t1 and t2.

26. The method according to claim 23, wherein the geometric eye model includes a first distance model, a second distance model, and a third distance model, wherein the first distance model describes a distance between the laser feedback interferometry unit and the retina, wherein the second distance model describes a distance between the laser feedback interferometry unit and the iris, wherein the third distance model describes a distance between the laser feedback interferometry unit and the sclera, wherein the following applies to the first distance model: d=d_{Re}(r_{Retina}), where d is the distance component, S is a position of the laser feedback interferometry unit, L is the laser signal and r_{Retina} is a radial distance between the retina and an eye center, wherein the following applies to the third distance model: d=d_{Sc}(r_{Sclera}), where r_{Sclera} is a radial distance between the sclera and the eye center, and wherein the following applies to the second distance model:
$$d = \frac{( {R}_{p} {e}_{0} - S ) \cdot ( {R}_{p} e )}{L \cdot ( {R}_{p} e )} ,$$
wherein Rₚ is a rotation matrix of the iris, e₀ is a unit vector from the eye center to the iris, e is a unit vector of an iris surface and L is a vector representation of the laser signal.

27. The method according to claim 16, wherein the ascertained eye position is a future eye position that the eye will assume at a predetermined future time point based on the determined rotational velocity.

28. The method according to claim 17, wherein the at least two laser interferometry units are arranged relative to the eye in such a way that eye rotational movements about at least two axes of rotation arranged perpendicularly to one another can be determined, and wherein rotational movements of the eye about the at least two axes of rotation arranged perpendicularly to one another can describe every physiologically possible eye position of a human eye.

29. A computing unit configured to determine an eye position, the computing unit configured to:
receive laser feedback interferometry measurement values of a laser feedback interferometry measurement of an eve using at least one laser interferometry unit, wherein the laser feedback interferometry measurement values are based on at least one laser signal reflected on a component of the eye;
determine a velocity component of the component of the eye relative to the laser feedback interferometry unit based on the laser feedback interferometry measurement values;
determine a rotational velocity of the eye about an axis of rotation based on the velocity component by using a geometric eye model, wherein the geometric eye model describes a function between the velocity component and the rotational velocity about the axis of rotation;
ascertain an eye position based on the rotational velocity about the axis of rotation by integrating the rotational velocity over a predetermined time segment by taking into account a known eye position as a starting point of the integration; and
provide the eye position change.

30. A non-transitory computer readable medium on which is stored a computer program including instructions for determining an eye position, the instructions, when executed by a data processor, causing the data processor to perform the following steps:
receiving laser feedback interferometry measurement values of a laser feedback interferometry measurement of an eye using at least one laser interferometry unit, wherein the laser feedback interferometry measurement values are based on at least one laser signal reflected on a component of the eye;
determining a velocity component of the component of the eye relative to the laser feedback interferometry unit based on the laser feedback interferometry measurement values;
determining a rotational velocity of the eye about an axis of rotation based on the velocity component by using a geometric eye model, wherein the geometric eye model describes a function between the velocity component and the rotational velocity about the axis of rotation;
ascertaining an eye position based on the rotational velocity about the axis of rotation by integrating the rotational velocity over a predetermined time segment by taking into account a known eye position as a starting point of the integration; and
providing the eye position change.
